(19) United States
(12) Patent Application Publication
(10) Pub. No.: US 2006/0270626 A1
(43) Pub. Date: Nov. 30, 2006

(54) Crude exopolysaccharides produced from phellinus baumii mycelium having hypoglycemic activity and preparation method thereof

(76) Inventors: Hye Jin Hwang, Daegu (KR); Sang Woo Kim, Daegu (KR); Jong Won Yun, Daegu (KR); Jang Won Choi, Daegu (KR)

Correspondence Address: HARNESS, DICKEY & PIERCE, P.L.C., P.O. BOX 828, BLOOMFIELD HILLS, MI, 48303, US

(21) Appl. No.: 11/414,718

(22) Filed: Apr. 28, 2006

(30) Foreign Application Priority Data
May 26, 2005 (KR) 10-2005-0043826

(51) Int. Cl. A61K 31/715; C08B 37/00

(52) U.S. Cl. 514/54; 536/123

(57) ABSTRACT

The present invention relates to a crude exopolysaccharides (EPS) produced from Phellinus baumii mycelium comprising mannose and/or arabinose and a preparation method thereof.

### CROSS-REFERENCE TO RELATED APPLICATION

This application claims priority to and the benefit of Korea Patent Application No. 2005-0043826, filed on May 24, 2005, the entire content of which is incorporated herein by reference.

### BACKGROUND OF THE INVENTION

1. Field of the Invention

This invention relates to crude exopolysaccharides produced from Phellinus baumii mycelium having hypoglycemic activity and preparation method thereof. Especially this invention develops a method to extract exopolysaccharides from submerged mycelial cultures of Phellinus baumii, investigates the effect of the extracted exopolysaccharides on the plasma glucose level in streptozotocin (STZ)-induced diabetic rats, and therefore suggests an use of exopolysaccharides for hypoglycemic medicine.

2. Description of the Related Art

Though different types of oral hypooglycelnic agents are available along with insulin for the treatment of diabetes mellitus, there is increasing demand by patients to use the natural ones without any side effects. Because insulin cannot be used orally and continuous use of the synthetic antidiabetic medicine causes side effects and toxicity (Yamamoto et al., Cytotoxicity and apoptosis produced by troglitazone in human hepatoma cells, LIFE SCIENCES 70, 471-482, 2001). Therefore, various kinds of natural products such as herbal drugs are widely prescribed even though their biological activation compounds are unknown because of their effectiveness, limited side effect, and relatively lower cost comparing to synthetic antidiatic medicine (Li et al., Natural medicines used in the traditional Chinese medical system for therapy of diabetes mellitus, JOURNAL OF ETHNOPHARMACOLOGY 92,1-21, 2004).

With thousand years of medical practice, a great deal of valuable experience has been accumulated in the traditional Chinese medical system for diabetes therapy. To date, there are about 33 traditional Chinese medicines that are most frequently used in traditional Chinese prescriptions for the clinical treatment of diabetes and its complications. The ailti-diabete bioactive compounds in Chinese medicinal plants include polysaccharides, terpenolds, flavonoids, sterols and alkanoids (Li et al Natural medicines used in the traditional Chinese medical system for therapy of diabetes mellitus, JOURNAL OF ETHNOPHARMACOLOGY 92, 1-21, 2004). In many countries, much attention has been paid to discover natural antidiabetic medicines from various medicinal plants (Ugochukwu et al., Antihyperglycemic effect of aqueous and ethanolic extracts of Gongronema latifolium leaves on glucose and glycogen metabolism in livers of normal and streptozotocin-induced diabetic rats, LIFE SCIENCES 73, 1925-1938, 2003).

Mushrooms and enthomopathogenic fungi are also exemplary sources of natural medicines with antidiabetic activity. With little scientific evidence, many researchers have endeavored to study the hypoglycemic effect from ether the fruiting body or mycelia of various edible/medicinal fungi including Tremella aurantia, Cordyceps sinensis, and Lentinus edodes (Kiho et al., Polysaccharide in fungi. XXXV, Anti diabetic activity of an acidic polysaccharide from the fruiting bodies of Tremella aurantia, BIOLOGICAL & PHARMACEUTICAL BULLETIN 18, 1627-1629, 1995; Kiho et al., Polysaccharide in fungi, XXXVI, Hypoglycemic activity of a polysaccharide (CS-F30) from the cultural mycelium Cordyceps sinensis and its effect on glucose metabolism in mouse liver. BIOLOGICAL & PHARMACEUTICAL BULLETIN 19, 294-296, 1996;Yang et al., Hypoglycemic effect of a Lentinus edodes exo-polymer produced from a submerged mycelial culture, BIOSCIENCE BIOTECHNOLOGY AND BIOCHEMISTRY 66, 937-942, 2002). Lo et al. reported that the fruiting body of Cordyceps has a potential to be a functional food for diabetes in 2004 (Lo et al., The anti-hyperglycemic activity of the fruiting body of Cordyceps in diabetic rats induced by nicotinamide and streptozotocin, LIFE SCIENCES 74, 2897-2908, 2004). Kiho et al. stated that the hot water extract of the fruiting body of Agrocybe cylindracea also has hypoglycemic effect in 1994 (Kiho et al., Structural features and hypoglycemic activities of two polysaccharides from a hot-water extract of Agrocybe cylindracea. Carbohydrate Research 251, 81-87, 1994). Yuan et al. investigated the hypoglycemic activity of a water-soluble polysaccharide from the fruiting bodies of Auricularia auricula-judae Quel in 1998 (Yuan et al., Hypoglycemic effect of water-soluble polysaccharide from Auricularia auricula-judae Quel. on genetically diabetic KK-A^{y} mice, BIOSCIENCE, BIOTECHNOLOGY, AND BIOCHEMISTRY 62, 1898-1903, 1998). Nevertheless, for this purpose, little effort has been made to use EPS obtained by mycelial culture of medicinal mushrooms (Balon et al., A fermentation product of Cordyceps sinensis increases whole-body insulin sensitivity in rats. THE JOURNAL OF ALTERNATIVE & COMPLEMENT MEDICINE 8, 315-323, 2002).

On the other hand, Phellinus baumii together with Phellinus linteus, is a mushroom used as a folk medicine for a variety of human diseases in several Asian countries. Jang et al. reported that extract of Phellinus baumii might be useful for preventing acute pneumonia in human diseases in 2004 (Jang et al., Extracts of Phellinus gilvus and Phellinus baumii inhibit pulmonary inflammation induced by lipopolysaccharide in rats, BIOTECHNOLOGY LETTERS 26, 31-33, 2004). The antioxident and free radical scavenging activity of Phellinus baumii extract were also examined by Shon et al. in 2003 (Shon et al., Antioxidants and free radical scavenging activity of Phellinus baumii (Phellinus of Hymenochaetaceae) extracts, FOOD CHEMISTRY 82, 593-597, 2003). To investigate the biological activities, most researchers have used the fruiting body of Phellinus baumii rather than EPS obtained from submerged mycelial culture broth. And there is no report on the hypoglycemic effect of EPS obtained from mycelial cultures of Phellinus baumii.

Therefore, the objective of the present invention is to provide the crude exopolysaccharides (EPS) produced by submerged mycelial culture of Phellinus baumii mycelium, which has the powerful hypoglycemic effect.

And the other objective of the present invention is to provide a method to produce the said crude exopolysaccharides (EPS) from submerged mycelial culture of Phellinus baumii mycelium.

### SUMMARY OF THE INVENTION

The present invention provides crude exopolysaccharides produced from mycelial culture of Phellinus baumii mycelium, which is compounded of more than two kinds of different heteropolysaccharides and more than two kinds of proteoglycans, and consists of protein moiety containing arginine and glycine, and carbohydrirate moiety containing mannose and arabinose.

The present invention provides the antidiabetic agent that contains effective component of said heteropolysaccharides (EPS).

The present invention provides the liver function improving agent that contains effective component of said heteropolysaccharides (EPS).

And the present invention provides a production method of exopolysaccharides (EPS) including the crude exopolysaccharides (EPS) produced from Phellinus baumii mycelium as mentioned on [0009] above, which comprises preparation of submerged culture filtration of mycelia; addition of alcohol into the said filtration; centrifugal separation of the said filtration; freeze dry of supernatant from the said filtration.

### BRIEF DESCRIPTION OF THE DRAWINGS

FIG. 1 shows the elution profile of the crude exopolysaccharides from Phellinus baumii in Sepharose CL-4B chromatography. Each fraction was noted as numbers 1, 2, 3 and 4.

FIG. 2 shows the effect of exopolysaccharides from Phellinus baumii on the plasma glucose level in STZ-induced diabetic rats.

### DETAILED DESCRIPTION

The present invention is configured with several steps to obtain EPS from submerged mycelial culture broth of Phellinus baumii, to measure the plasma glucose, cholesterol and triglyceride level, and determine the activities of alamine aminotransferase (ALT) and asperate aminotransferase (AST).

The present invention is to provide EPS having hypoglycemic activity which is produced from submerged mycelial culture broth of Phellinus baumii, compounded of two (2) heteropolysaccharides and two (2) proteoglicans, and consists of arginine and glycine in protein moiety, and mannose and arabinose in carbohydrirate moiety.

The production of the invention, EPS extracted from myclial culture broth of Phellinus baumii can be obtained through several steps to get mycelial pellet from the submerged culture filtrate mixed with four volumes of absolute ethanol, stirred vigorously and left overnight at 4° C., to dissolve the pellet into 0.2M NaCl buffer to a concentration of 10 g/l, and to elute EPS loaded onto Separose CI-4B column (2.4 cm×100 cm).

To investigate the hypoglycemic effect of EPS produced from myclial culture broth of Phellinus baumii, diabetic rats were treated with Phellinus baumii EPS at the level of 200 mg/kg body weight using an oral zoned.

The present invention will be described in detail by referring to the following Examples, but the technical scope of the present invention is not limited by these Examples.

### EXAMPLES

### Example I

### Production of EPS From Mycelial Culture Broth of Phellinus baumii

The Phellinus baumii materials to produce EPS with hypoglycemic effect from Phellinus baumii had been extracted from the fresh mycelia by the inventors (Laboratory of Functional Materials, Department of Biotechnology, Daegu University, Kyungbuk, Korea). The stock culture was inoculated on a potato dextrose agar (PDA) slant, incubated at 28□ for six (6) days, and used throughout the experiment. The stock culture was maintained by monthly subculture and the slants were stored at 4□.

To prepare inoculum, Phellinus baumii was initially grown on a PDA medium (2.4% potato dextrose broth and 2% agar) in a petri dish, and then transferred to the seed culture medium by punching out a portion (5 mm diameter) of the agar plate with a sterilized house-developed cutter. The seed culture was grown in a 250 ml flask containing 50 ml of PMP medium (2.4% potato dextrose broth, 1% malt extract, 0.1% peptone) at 28□ with shaking at 150 rpm for 4 days.

The submerged culture of Phellinus baumii to produce EPS was operated in a 5L stirred-type fermentation tank under the following culture conditions: fructose 20 g/L, yeast extract 20 g/L, CaCl₂ 0.55 g/L; temperature, 30□; aeration rate, 2 vvm; agitation speed, 150 rpm; initial pH, 5.0; working volume, 3L.

To extract EPS, the culture broths were centrifuged at 10,000×g for 20 minutes, and then the resulting supernatant was filtered through a Whatman filter paper No. 2 (Whatman International Ltd., Maidstone, England). The resulting culture filtrate was mixed with four volumes of absolute ethanol, stirred vigorously and left overnight at 4□. The precipitated EPS was centrifuged at 10,000×g for 20 min and the supernatant was discarded. The precipitates of crude EPS were lyophilized and the weight was estimated. Dry weight of the mycelium was measured after repeated washing of the mycelial pellet with distilled water and drying at 90□ overnight to a constant weight.

The ethanol precipitates of the crude EPS components were dissolved in 0.2 M NaCl buffer to a concentration of 10 g/L, and loaded onto a Sepharose CL-4B column (2.4 cm×100 cm, Sigma Chemical Co., Louis, Mo., USA). The column was eluted with the same buffer at a flow rate of 0.6 mL/min. The total carbohydrate content of the EPS produced from Phellinus baumii was determined by phenol sulfuric acid method (Dubois et al., Colorimetric method for determination of sugar and related substance, ANALYTICAL CHEMISTRY 28, 350-356, 1956) using glucose as the standard. Total protein was determined by the Lowry method (Lowry et al., 1951. Protein measurement with the folin phenol reagent, JOURNAL OF BIOLOGICAL CHEMISTRY 193, 265-275, 1951) with bovine serum albumin as the standard. The protein moiety in the EPS was monitored by absorbance at 280 nm, whilst the carbohydrate moiety was monitored at 480 nm.

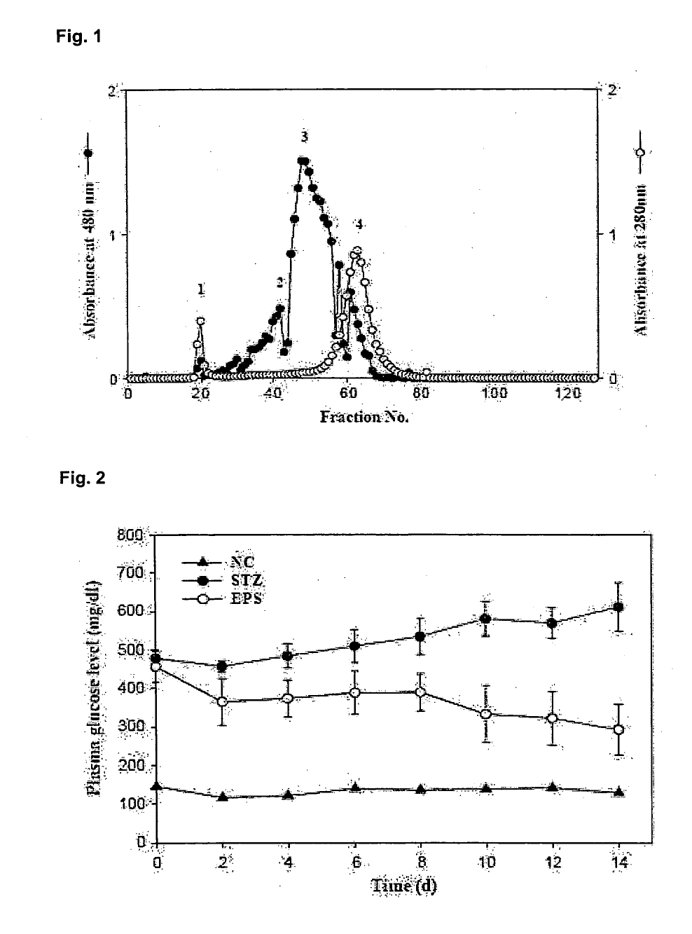

When mycelial growth and EPS production were achieved in a 5 L stirred-type fermentation tank, the maximum mycelial concentration reached about 20 g/L on day 14, whereas the maximum EPS production (about 5 g/L) was achieved on day 16 (data not shown in FIG. 1).

The crude EPS was monitored by a gel filtration in a Sepharose CL-4B column, by which two different heteropolysaccharides and two proteoglycans were eluted as shown in FIG. 1. The carbohydrate and protein contents in the crude EPS were 71.0% and 29.0%, respectively. The crude EPS consisted of mainly arginine (14.1%) and glycine (12.0%) in protein moiety and mainly mannose (48.7%) and arabinose (38.4%) in carbohydrate moiety (data not shown in FIG. 1).

### Example 2

### Investigation on Hypoglycemic Effect of the EPS Produced From Phellinus baumii Mycelium

The present invention used a STZ-induced diabetic rat model to investigate the hypoglycemic effect of Phellinus baumii EPS. Streptozotocin (STZ; N-nitroso derivative of glucosamine) is a broad-spectrum antibiotic extracted from Streptomyces acromogenes. It is a pancreatic β-cells toxin that induces rapid and irreversible necrosis of β-cells and is widely used to induce IDDM in experimental animal models (Junod et al., Studies of the diabetogenic action of streptozotocin, PROCEEDING OF THE SOCIETY FOR EXPERIMENTAL BIOLOGY AND MEDICINE 126, 201-205, 1967; Andrade-Cetto et al., Hypoglycemic effect of Cecropia obtusifolia on streptozotocin diabetic rats, JOURNAL OF ETHNOPHARMACOLOGY 78, 145-149, 2001; Benwahhoud et al., Hypoglycemic effect of Suaeda fruticosa in streptozotocin-induced diabetic rats, JOURNAL OF ETHNOPHARMACOLOGY 76, 35-38, 2001).

Male Sprague-Dawley rats (Daehan Experimental Animals, Seoul, Korea), weighing 130-150 g, were housed in individual stainless steel cages and acclimatized with free access to food and water for at least one (1) week in an air conditioned room (23±2□ with 55±5% humidity) under a 12:12-hour light-dark cycle. The rats were fed with a commercial pellet diet (Samyang Co., Seoul, Korea) throughout the experimental period.

After one week of acclimatization, the rats were subjected to a 16-hour fast. Diabetes was induced by intramuscular injection of streptozotocin (Sigma Chemical Co., Louis, Mo., USA) dissolved in 0.01 M sodium citrate buffer (pH 4.5) at a dose of 50 mg/kg body weight (Bolkent et al., Effects of chard (beta vulgaris L. var cicla) extract on pancreatic β-cell in STZ-diabetic rats: a morphological and biochemical study, JOURNAL OF ETHNOPHARMACOLOGY 73, 251-259, 2000; Kim et al., Production of a hypoglycemic, extracellular polysaccharide from the submerged culture of the mushroom, Phellinus linteus, BIOTECHNOLOGY LETTERS 23, 513-517, 2001). Two days after injection of diabetogenic agent (STZ), fasting blood glucose was determined and the rats with more than blood glucose >300 mg/dL were included in the group of diabetics.

All the animals were randomly divided into three groups with six animals in each group: normal control group (NC), normal rats received 0.9% NaCl solution; STZ-induced diabetic control group (STZ), diabetic rats treated with 0.9% NaCl solution; diabetic treated group (EPS), diabetic rats treated with Phellinus baumii EPS at the level of 200 mg/kg body weight using an oral zoned daily for 14 days.

On the basis of the preliminary test result (doses of 100 and 200 mg/kg) and other experimental designs by several investigators who used natural sources for diabetic therapy (normal dose ranges: 100-300 mg/kg), 200 mg/kg was chosen as a suitable dose for this experiment (Kim et al., Hypoglycemic effects of mycelia produced from a submerged culture of Phellinus linteus (Berk. et Curt) Teng (Aphyllophoromycetideae) in streptozotocin-induced diabetic rats, INTERNATIONAL JOURNAL OF MEDICINAL MUSHROOMS 3, 21-26, 2001; Kim. et al., A preliminary study on the hypoglycemic effect of the exo-polymers produced by five different medicinal mushrooms, JOURNAL OF MICROBIOLOGY AND BIOTECHNOLOGY 11, 167-171, 2001; Lo et al., The anti-hyperglycemic activity of the fruiting body of Cordyceps in diabetic rats induced by nicotinamide and streptozotocin, LIFE SCIENCES 74, 2897-2908, 2004; Benwahhoud et al., Hypoglycemic effect of Suaeda fruticosa in streptozotocin-induced diabetic rats, JOURNAL OF ETHNOPHARMACOLOGY 76, 35-38, 2001).

The body weight gain and food intake were periodically measured. Blood samples of the experimental animals were collected in tubes treated with 0.1 M EDTA as anticoagulant, and plasma was separated by centrifugation at 3000×g for 10 minutes. The plasma glucose, cholesterol, and triglyceride levels were measured using an enzymatic calorimetric assay (YD Diagnostics Co., Ltd., Seoul, Korea). The activities of alanine aminotransferase (ALT) and asparate aminotransferase (AST) were determined by using enzyme kits (YD Diagnostics Co., Ltd., Seoul, Korea) based on the Reitman-Frankel method (Reitman et al., A calorimetric method for the determination of serum glutamic oxaloacetic and glutamic pyruvic transaminase, AMERICAN JOURNAL OF CLINICAL PATHOLOGY 28, 56-63, 1957).

The results were analyzed for statistical significance by one-way analysis of variance (ANOVA) test using the Statistical Package of the Social Science (SPSS) software program. All data were expressed as mean ±S.E (P<0.05). Group means were considered to be significantly different at P<0.05, as determined by the technique of protective least-significant difference (LSD).

The administration effect of Phellinus baumii EPS on the body weight gain and food intake in STZ-induced diabetic rats is presented in Table 1 as below.

TABLE 1
Effect of Phellinus baumii exopolysaccharides
(EPS) on the body weight gain and food intake
in STZ-induced diabetic rats for 14 days.

| | Body weight gain | Food intake | Food efficiency |
|---|---|---|---|
| Group^{a} | (g/day) | (g/day) | ratio^{b} |
| NC | 5.19 ± 0.32^{c} | 14.53 ± 0.71 | 0.36 ± 0.02 |
| STZ | 2.90 ± 0.50* | 18.62 ± 0.64* | 0.15 ± 0.02* |
| EPS | 3.83 ± 0.44* | 15.59 ± 0.71** | 0.25 ± 0.03* ** |

^{a}For detail group division
^{b}Body weight gain/Food intake
^{c}Values are means ± S.E. (n = 6)
*Significantly different from NC group, P < 0.05
**Significantly different from STZ group, P < 0.05

As shown in Table 1, a significant difference in both body weight gain and food intake was observed between diabetic groups (STZ and EPS) and nondiabetic animals (NC). The food intake amount of the diabetic control rats (STZ) increased by 28.1%, whereas body weight gain was reduced by about 44.1% as compared to the normal control rats (NC). Moreover, rats in STZ also had a markedly lower food efficiency ratio than those in the NC and EPS groups. The cause of intake suppression may be due to the presence of a specific compound in EPS that has not yet been elucidated (Kiho et al., Antidiabetic effect of an acidic polysaccharide (TAP) from Tremella aurantia and its degradation product (TAP-H), BIOLOGICAL & PHARMACEUTICAL BULLETIN 24, 1400-1403, 2001).

FIG. 2 shows the effect of Phellinus baumii EPS on the plasma glucose level in STZ-induced diabetic rats over a 14 day period. In the two diabetic groups (STZ and EPS), the concentrations of plasma glucose baseline were almost the same, with both groups within the error range. In the diabetic control group (STZ), the plasma glucose level continuously increased during the experimental period, reaching a final level of 610 mg/L. In contrast, the administration of Phellinus baumii EPS significantly lowered the plasma glucose level by 52.3% on day 14 when compared to that of the diabetic control group.

TABLE 2
Effect of Phellinus baumii exopolysaccharides (EPS)
on the plasma total cholesterol and triglyceride
in STZ-induced diabetic rats for 14 days

| Group^{a} | Total cholesterol (mg/dL) | Triglyceride (mg/dL) |
|---|---|---|
| NC | 98.40 ± 6.09^{b} | 52.64 ± 4.27 |
| STZ | 92.13 ± 5.66 | 99.30 ± 7.72* |
| EPS | 102.04 ± 4.70 | 60.14 ± 5.21** |

^{a}For detail group division
^{b}Values are means ± S.E. (n = 6)
*Significantly different from NC group, P < 0.05
**Significantly different from STZ group, P < 0.05

As shown in Table 2, there was no significant difference in plasma total cholesterol level among the groups during the experimental period. The plasma triglyceride level was markedly reduced by oral administration of Phellinus baumii EPS.

Generally, the plasma AST and ALT levels increase as a result of metabolic changes in the liver, such as administration of toxin, cirrhosis of the liver, hepatitis, and liver cancer (Bursch et al., Cytoprotective effect of the prostacyclin derivatitve iloprost against liver cell death induced by the hepatotoxins carbon tetrachloride and bromobenzene, KLINISCHE WOCHENSCHRIFT 64, 47-50, 1986; Chalasani et al., Patients with elevated liver enzymes are not at higher risk for statin hepatotoxicity, GASTROENTEROLOGY 126, 1287-1292, 2004; Dieterich, Managing antiretroviral-associated liver disease, JOURNAL OF ACQUIRED IMMUNE DEFICIENCY SYNDROMES 34, S34-S39, 2003). Thus, they can be used as markers to assess the extent of liver damage. Table 3 shows the administration effects of Phellinus baumii EPS on the plasma AST and ALT activities in STZ-induced diabetic rats.

TABLE 3
Effect of Phellinus baumii exopolysaccharides (EPS) on the
plasma AST and ALT in STZ-induced diabetic rats for 14 days

| Group^{a} | AST (Karmen unit/L)^{b} | ALT (Karmen unit/L/dL) |
|---|---|---|
| NC | 94.49 ± 3.54^{c} | 43.13 ± 2.07 |
| STZ | 118.65 ± 13.23* | 78.52 ± 11.69* |
| EPS | 97.82 ± 3.82 | 50.25 ± 3.13** |

^{a}For detail group division
^{b}Karmen unit means a formerly used enzyme unit for aminotransferase activity; a change of 0.001 in the absorbance of NADH/min.
^{c}Values are means ± S.E. (n = 6)
*Significantly different from NC group, P < 0.05
**Significantly different from STZ group, P < 0.05

As shown on Table 2, in comparison with the NC group, the plasma AST and ALT activities in the STZ group were increased by 25.6% and 82.1%, respectively. Therefore, the remarkable reduction of AST and ALT levels upon oral administration of Phellinus baumii EPS exhibited a remedial role on liver function.

And the variances in weight of the organs for the experimental rats were determined to indirectly diagnose the diabetes. Table 4 shows the variances in weight of the organs for the three experimental groups.

TABLE 4
Effect of Phellinus baumii exopolysaccharides (EPS) on the weight
of the various organs in STZ-induced diabetic rats for 14 days

| | Organs weight (g/100 g Body Weight) | | | | | |
|---|---|---|---|---|---|---|
| Group^{a} | Liver | Heart | Spleen | Kidney | Pancreas | Lung |
| NC | 4.18 ± 0.03^{c} | 0.36 ± 0.01 | 0.26 ± 0.01 | 0.71 ± 0.12 | 0.28 ± 0.02 | 0.44 ± 0.01 |
| STZ | 4.93 ± 0.11* | 0.40 ± 0.01 | 0.40 ± 0.05* | 1.15 ± 0.29* | 0.30 ± 0.02 | 0.59 ± 0.04* |
| EPS | 4.54 ± 0.12* ** | 0.38 ± 0.01 | 0.37 ± 0.02* | 0.88 ± 0.57* ** | 0.29 ± 0.02 | 0.60 ± 0.04* |

^{a}For detail group division
^{b}Values are means ± S.E. (n = 6)
*Significantly different from NC group, P < 0.05
**Significantly different from STZ group, P < 0.05

As shown on Table 4, there were no significant differences in the weights of the heart and pancreas, whereas those of the liver, spleen, kidney, and lung were considerably increased in the diabetic groups (STZ and EPS) as compared to the NC group.

### Comparison Example 1

### Comparison of Hypoglycemic Effects of Various Mushrooms in Diabetic Animals

Most drugs for therapy of diabetes mellitus have focused on controlling and lowering blood glucose to a normal level (Li et al., Natural medicines used in the traditional Chinese medical system for therapy of diabetes mellitus, JOURNAL OF ETHNOPHARMACOLOGY 92, 1-21, 2004). Hypoglycemic effects of various mushrooms in literature were reviewed and summarized in Table 5. The hypoglycemic effects were estimated as the reduction ratio of blood glucose level in experimental groups to that of the diabetic control group.

TABLE 5
Hypoglycemic effects of various mushrooms in diabetic animals

| | | Diabetic | Hypoglycemic | |
|---|---|---|---|---|
| Mushrooms | Materials (Methods)^{a} | animals^{b} | effect (period)^{c} | References |
| Agaricus | Extract of fruiting body (DW) | Mice (STZ) | 50.4 (12 d) | Gray et al., 1998 - 1) |
| | and fruiting body powder (FD) | | | |
| Auricularia uricula-judae | Extract of fruiting body (FD) | KK-A^{y}-TA mice | 50.5 (14 d)^{d} | Yuan et al., 1998 - 2) |
| Quel. | | | | |
| Cordyceps militaris | Extract of fruiting body (OA) | SD rat (STZ) | 21.4 (16 h) | Kwon et al., 2001 - 3) |
| Cordyceps militaris | EPS (OA) | SD rat (STZ) | 14.0 (7 d) | Kim et al., 2001 - 4) |
| Cordyceps sinensis | Extract of mycelia (IP) | KK-A^{y}-TA mice | 13.7 (48 h) | Kiho et al., 1996 - 5) |
| Cordyceps sinensis | Extract of mycelia (OA) | ddy mice (STZ) | 15.0 (24 h) | Kiho et al., 1993 - 6) |
| Ganoderma lucidum | Extract of soybean paste containing mycelia (OA) | SD rat (STZ) | 44.6 (7 d) | Yang et al., 2000 - 7) |
| Grifola frondosa | Extract of fruiting body (OA) | KK-A^{y}-TA mice | 54.0 (14 d)^{d} | Kubo et al., 1994 - 8) |
| Lentinus endodes | EPS (OA) | SD rat (STZ) | 21.2 (7 d) | Yang et al., 2002 - 9) |
| Lentinus endodes | Extract of mycelia (OA) | SD rat (STZ) | 31.7 (14 d) | Kim et al., 1997 - 10) |
| Lentinus endodes | Mycelia powder (FD) | SD rat (STZ) | 23.0 (7 d) | Yang et al., 2002 - 11) |
| Phellinus baumii | EPS (OA) | SD rat (STZ) | 52.3 (14 d) | This invention |
| Phellinus linteus | EPS (OA) | SD rat (STZ) | 49.0 (7 d) | Kim et al., 2001 - 12) |
| Phellinus linteus | Mycelia powder (FD) | SD rat (STZ) | 28.0 (7 d) | Kim et al., 2001 - 13) |
| Pleurotus eryngii | Mycelia powder (FD) | SD (STZ) | 16.9 (14 d) | Kim et al., 2001 - 14) |
| Tremella aurantia | Extract of fruiting body (IP) | KK-A^{y}-TA mice | 30.9 (24 h) | Kiho et al., 1995 - 15) |

^{a}DW: drink water; FD: feeding with diet; IP: intraperitoneal administration; OA: oral administration
^{b}ddy mice (STZ), and SD rat (STZ): diabetic animals induced by the injection of streptozotocin for the induction of type 1 diabetes; KK-A^{y}-TA mice: one of the animal model for type 2 diabetes.
^{c}Hypoglycemic effect (%) was estimated as the reduction ratio of blood glucose level to that of diabetic control.
^{d}The data indicate approximate values calculated by the authors from figures in the references.
1) Gray, A. M., Flatt, P. R., Insulin-releasing and insulin-like activity of Agaricus campestris (mushroom), Journal of Endocrinology 157, 259-266, 1998.
2) Yuan, Z., He, P., Cui, J., Takeuchi, H., Hypoglycemic effect of water-soluble polysaccharide from Auricularia auricula-judae Quel. on genetically diabetic KK-A^{y} mice, BIOSCIENCE, BIOTECHNOLOGY, AND BIOCHEMISTRY 62, 1898-1903, 1998.
3) Kwon, Y. M., Cho, S. M., Kim, J. H., Lee, J. H., Lee, Y. A., Lee, S. J., Lee, M. W., Hypoglycemic effect of Cordyceps militaris, KOREAN JOURNAL OF PHARMACOGNOSY 32, 327-329, 2001.
4) Kim, D. H., Yang, B. K., Jeong, S. C, Hur, N. J., Das, S., Yun, J. W., Choi, J. W., Lee, Y. S., Song, C. H., A preliminary study on the hypoglycemic effect of the exo-polymers produced by five different medicinal mushrooms, JOURNAL OF MICROBIOLOGY AND BIOTECHNOLOGY 11, 167-171, 2001.
5) Kiho, T., Yamane, A., Hui, J., Usui, S., Ukai, S., Polysaccharide in fungi, XXXVI, Hypoglycemic activity of a polysaccharide (CS-F30) from the cultural mycelium Cordyceps sinensis and its effect on glucose metabolism in mouse liver, BIOLOGICAL & PHARMACEUTICAL BULLETIN 19, 294-296, 1996.
6) Kiho, T., Hui, J., Yamane, A., Ukai, S., Polysaccharides in Fungi. XXXII, Hypoglycemic activity and chemical properties of a polysaccharide from the cultural mycelium of Cordyceps sinensis, BIOLOGICAL & PHARMACEUTICAL BULLETIN 16, 1291-1293, 1993.
7) Yang, B. K., Jeong, S. C., Hur, N. J., Ha, S. O., Kim, K. Y., Kym, K. H., Yun J. W., Song, C. H., Hypoglycemic effect of extracts of soybean paste containing mycelia of mushrooms in streptozotocin-induced diabetic rats, THE KOREAN JOURNAL OF MYCOLOGY 28, 126-129, 2000.
8) Kubo, K., Aoki, H., Nanba, H., 1994. Anti-diabetic activity present in the fruit body of Grifola frondosa (Maitake) I, BIOLOGICAL & PHARMACEUTICAL BULLETIN 17, 1106-1110
9) Yang, B. K., Kim, D. H., Jeong, S. C., Das, S., Choi, Y. S., Shin, J. S., Lee, S.C., Song C. H., Hypoglycemic effect of a Lentinus edodes exo-polymer produced from a submerged mycelial culture, BIOSCIENCE, BIOTECHNOLOGY, AND BIOCHEMISTRY 66, 937-942, 2002.
10) Kim, M. W., Park, M. H., Kim, G. H., Effects of mushroom protein-bound polysaccharides on blood glucose levels and energy metabolism in streptozotocin-induced rats, THE KOREAN JOURNAL OF NUTRITION 30, 743-750, 1997.
11) Yang, B. K., Kim, D. H., Song, C. H., Production of Lentinus edodes mycelia in submerged culture and it's hypoglycemic effect in diabetic rats, THE KOREAN JOURNAL OF MYCOLOGY 30, 131-135, 2002.
12) Kim, D. H., Yang, B. K., Jeong, S. C., Park, J. B., Cho, S. P., Das, S., Yun, J. W., Song, C. H., Production of a hypoglycemic, extracellular polysaccharide from the submerged culture of the mushroom, Phellinus linteus, BIOTECHNOLOGY LETTERS 23, 513-517, 2001.
13) Kim, D. H., Yang, B. K., Hur, N. J., Das, S., Yun, J. W., Choi, Y. S., Song, C. H., Hypoglycemic effects of mycelia produced from a submerged culture of Phellinus linteus (Berk. et Curt) Teng (Aphyllophoromycetideae) in streptozotocin-induced diabetic rats, INTERNATIONAL JOURNAL OF MEDICINAL MUSHROOMS 3, 21-26, 2001.
14) Kang, T. S., Kang, M. S., Sung, J. M., Kang, A. S., Shon, H. R., Lee, S. Y., Effect of Pleurotus eryngii on the blood glucose and cholesterol in diabetic rats, THE KOREAN JOURNAL OF MYCOLOGY 29, 86-90, 2001.
15) Kiho, T., Morimoto, H., Sakushima, M., Usui, S., Ukai, S., Polysaccharide in fungi. XXXV, Anti diabetic activity of an acidic polysaccharide from the fruiting bodies of Tremella aurantia, BIOLOGICAL & PHARMACEUTICAL BULLETIN 18, 1627-1629, 1995.

As shown in Table 5, the hypoglycemic effect of EPS was generally lower than those of extracts from mycelia or fruiting bodies of mushrooms. In the case of type 1 diabetic animals, the hypoglycemic effect of Phellinus baumii EPS was the highest even though the difference was not so significant compared to that of the fruiting body extract of Agaricus campestris. It is noteworthy that the highest hypoglycemic effect (54.0%) was achieved with the fruiting body extract of Grifola frondosa in type 2 diabetic animals. For the purpose of medical practice, it is not feasible to employ the fruiting body because it requires a complicated extraction process and is difficult to obtain uniform chemical composition. In this regard, it is more reasonable to use EPS obtained from mycelial cultures rather than fruiting body extracts of mushrooms.

Based on the results above, the present invention found that Phellinus baumii EPS administration led to the diabetogenic effect of STZ and significantly reduced the degree of diabetes. Therefore, the inventors of the present invention beleve that oral administration of Phellinus baumii EPS may have a potential benefit in preventing diabetes, since pancreatic damage induced by environmental chemicals and other factors is a cause of diabetes (Lo et al., The anti-hyperglycemic activity of the fruiting body of Cordyceps in diabetic rats induced by nicotinamide and streptozotocin, LIFE SCIENCES 74, 2897-2908, 2004). However, the present invention needs further study to identify the active fractions responsible for hypoglycemic activity and to clarify the mechanism of the effect.

As mentioned on the examples above, the present invention relates to crude exopolysaccharides produced from Phellinus baumii mycelium having hypoglycemic activity and preparation method thereof. When the crude exopolysaccharide produced from Phellinus baumii mycelium medicates for the streptozotocin (STZ)-induced diabetic rats, it affects reducing the plasma glucose level in blood substantially. And the activities of alanine aminotransferase (ALT) and asparate aminotransferase (AST) are significantly decreased by operation of the invented EPS, thereby exhibiting a remedial role in liver function. Consequently the EPS of the present invention would be very useful invention in the medicinal industry for hypoglycemic control of diabetic patient and improving the reduced function of liver from diabetes.

Although the present invention has been described with reference to certain exemplary embodiments, it will be understood by those skilled in the art that a variety of modifications and variations may be made to the present invention without departing from the spirit or scope of the present invention defined in the appended claims, and their equivalents.

What is claimed is:

1. A Crude exopolysaccharides produced from Phellinus baumii mycelium comprising mannose and/or arabinose.

2. The crude exopolysaccharides of claim 1, wherein the crude exopolysaccharides has hypoglycemic activity.

3. The crude exopolysaccharides of claim 1, wherein the crude exopolysaccharides further comprises a protein.

4. The crude exopolysaccharides of claim 1, wherein the protein comprises arginine and/or glycine.

5. An antidiabetic agent containing crude exopolysaccharides produced from Phellinus baumii mycelium comprising mannose and/or arabinose as an effective component.

6. A liver function-improving agent containing a crude exopolysaccharides produced from Phellinus baumii mycelium comprising mannose and/or arabinose as an effective component.

7. A method of a crude exopolysaccharides produced from Phellinus baumii mycelium containing mannose and/or arabinose comprising;
Preparing a submerged culture of mycelia;
Filtering the submerged culture;
Adding alcohol into the filtration of the submerged culture;
centrifugal separating the filtration; and
freeze drying supernatant of the filtration.
